(19) United States
(12) Patent Application Publication
(10) Pub. No.: US 2024/0393281 A1
(43) Pub. Date: Nov. 28, 2024

(54) High-Accuracy Electrochemical Sensors for Instant Detection of Pathogens

(71) Applicant: Xiaoling Shi, Acton, MA (US)

(72) Inventor: Xiaoling Shi, Acton, MA (US)

(21) Appl. No.: 18/201,625

(22) Filed: May 24, 2023

(51) Int. Cl. G01N 27/12; C25D 9/02; G01N 33/543; G01N 33/569

(52) U.S. Cl. CPC G01N 27/125; C25D 9/02; G01N 33/5438; G01N 33/56983; G01N 2469/10

(57) ABSTRACT

A novel MIP electrochemical sensor for instant detecting pathogens from aerosol or liquid with high sensitivity and high specificity, the pathogens include SARS-COV-2, SARS-COV-2 spike proteins, RBD, MERS, flu viruses and Covid-19 viruses.

### CROSS-REFERENCE

Priority is claimed from the priority of U.S. Provisional Patent Application No. 63/323,320, titled “High-sensitivity and High-selectivity Electrochemical Sensors for Instant Detection of Pathogens” filed on Mar. 24, 2022, entirety of which is hereby incorporated by reference.

### DESCRIPTION OF RELATED ART

The present application relates to an electrochemical sensor for pathogens, and more particularly to an electrochemical sensor that is highly selective and highly sensitive to the detection of SARS-COV-2 virus.

Note that the points discussed below may reflect the hindsight gained from the disclosed inventions, and are not necessarily admitted being prior art.

Catastrophic pandemic, such as COVID-19, has disrupted military operations and communities' everyday lives. Existing methods for COVID-19 tests, such as PCR (polymerase chain reaction) assay testing of virus nucleic acids, are multiple-step and relatively complicated processes, slow, expensive, and inadequate. Reagents and equipment for such assays are very limited and time consuming, and the testing capacities are restricted by the complicated processes, even the testing capacities in US technology hubs such as Boston, Seattle, California, and New York are extremely inadequate to the demands in testing.

Also, in many situations, up to 50% of infections may not have any noticeable symptoms, but the carriers may still produce high-titer infectious viruses in the oropharynx even in the absence of symptoms. In addition, infectious virus such as SARS-CoV-2 may continue to be infectious after being in the air for hours or on surfaces for days. There is a great need for rapid and easy virus testing and identification for situation monitoring.

Alternative methods to PCR assay testing may also be required for ensuring pathogen-free delivery of military goods and for assisting the public to return to normal activities with peace of mind.

### SUMMARY

The present application discloses a novel and sensor-based approach to testing viruses, such as SARS-COV-2.

We developed ultra-compact gas sensors that will rapidly detect SARS-CoV-2 and other viruses in the air and on surfaces in 1˜2 seconds with ˜95% sensitivity and 95% specificity based on our demonstrated success.

The measurement of the volatile organic compounds (VOCs) produced by the body's metabolic activity is a powerful approach for health monitoring and disease detection. Our biomarker sensors are novel electrochemical gas sensors based on functionalized graphene/Prussian-blue and molecularly imprinted polymer (MIP) on a substrate for the diagnosis of different diseases from exhaled breath. A thin layer polymer is electrochemically deposited around the template molecules or particles to be measured, such as biomarker VOCs. The template molecules are then washed away after polymerization, leaving an imprint of the exact size and shape of the template molecule in the polymer matrix. The sensor only detects the originally targeted template molecules or particles due to the matched shape and size and the strong hydrogen bonding between the cavities on the sensors and the template molecules or particles, leading to extremely high specificity.

Our gas sensors can detect small molecules from the atomic level up to a larger scale, such as SARS-COV-2 virus particles with a size of ˜120 nm. We have successfully made and tested over 30 different sensors for different biomarker VOCs for the diagnosis of different diseases, including lung cancer, diabetes, Alzheimer's disease, drug abuse, and COVID-19. These biomarker VOC sensors all have an extremely high demonstrated limit of detection of part per quintillion (10-18) for oxycodone and respond to only the targeted molecules with very high specificity.

Other novel sensors having biomarker VOCs for other diseases can be developed using the established procedures described herein, following similar sensor design, fabrication, and tests in analytical and clinical environments.

The disclosed innovation, in various embodiments, provides one or more of at least the following advantages, however, not all of these advantages result from every one of the innovations disclosed, and this list of advantages does not limit the various claimed inventions.: high sensitivity and high selectivity, much simpler sample processing and equipment requirement, high scalability for large scale of sample testing.

### BRIEF DESCRIPTION OF THE DRAWINGS

The disclosed application will be described with reference to the accompanying drawings, which show important sample embodiments of the invention and which are incorporated in the specification hereof by reference, wherein:

FIG. 1A schematically shows the principle of the detection and structural mechanism of a biomaterial sensitized sensor in accordance with this application.

FIG. 1B schematically shows the step of generating a SARS-Cov-2 S-Protein sensor in accordance with this application.

FIG. 2A shows an example test tube setup for an example virus sensor in accordance with this application.

FIG. 2B shows an example detection equipment setup for an example virus sensor in accordance with this application.

FIG. 3 shows a testing result of one sample virus by an example virus sensor in accordance with this application.

FIG. 4 shows a testing result of another sample virus by the example virus sensor of FIG. 3 in accordance with this application.

FIG. 5 shows a testing result of another sample virus by the example virus sensor of FIG. 3 in accordance with this application.

FIG. 6 shows a testing result of another sample virus by the example virus sensor of FIG. 3 in accordance with this application.

FIG. 7 shows a testing result of another sample virus by the example virus sensor of FIG. 3 in accordance with this application.

FIG. 8 shows a testing result of another sample virus by the example virus sensor of FIG. 3 in accordance with this application.

FIG. 9 shows a testing result of another sample virus by the example virus sensor of FIG. 3 in accordance with this application.

FIG. 10 shows a testing result of another sample virus by the example virus sensor of FIG. 3 in accordance with this application.

FIG. 11 shows a testing result of another sample virus by the example virus sensor of FIG. 3 in accordance with this application.

FIG. 12 shows a testing result of another sample virus by the example virus sensor of FIG. 3 in accordance with this application.

FIG. 13 shows a testing result of another sample virus by the example virus sensor of FIG. 3 in accordance with this application.

FIG. 14 shows a testing result of another sample virus by the example virus sensor of FIG. 3 in accordance with this application.

FIG. 15 shows a testing result of another sample virus by the example virus sensor of FIG. 3 in accordance with this application.

FIG. 16 shows a testing result of another sample virus by the example virus sensor of FIG. 3 in accordance with this application.

FIG. 17 shows a testing result of another sample virus by the example virus sensor of FIG. 3 in accordance with this application.

FIG. 18 shows testing results of virous sample virus concentrations by another example virus sensor in accordance with this application.

FIG. 19 shows testing results of comparison between positive and negative samples in data analysis in accordance with this application.

FIG. 20 shows an image of a wafer coated with a biomarker VOC for sensing a particular virus in accordance with this application.

### DETAILED DESCRIPTION OF SAMPLE EMBODIMENTS

The numerous innovative teachings of the present application will be described with particular reference to presently preferred embodiments (by way of example, and not of limitation). The present application describes several embodiments, and none of the statements below should be taken as limiting the claims generally.

For simplicity and clarity of illustration, the drawing figures illustrate the general manner of construction, and description and details of well-known features and techniques may be omitted to avoid unnecessarily obscuring the invention. Additionally, elements in the drawing figures are not necessarily drawn to scale, some areas or elements may be expanded to help improve understanding of embodiments of the invention.

The terms “first,” “second,” “third,” “fourth,” and the like in the description and the claims, if any, may be used for distinguishing between similar elements and not necessarily for describing a particular sequential or chronological order. It is to be understood that the terms so used are interchangeable. Furthermore, the terms “comprise,” “include,” “have,” and any variations thereof, are intended to cover non-exclusive inclusions, such that a process, method, article, apparatus, or composition that comprises a list of elements is not necessarily limited to those elements but may include other elements not expressly listed or inherent to such process, method, article, apparatus, or composition.

It is contemplated and intended that the design apply to testing any types of biological samples that have corresponding identifiable biomarkers; for clarity reason, the examples are given based on constructions made with SARS-COV-2 virus particles; an ordinary person in the art would know the variations to modify the design to provide sensors for other biological and biomedical uses.

Volatile organic compounds (VOCs) are gaseous molecules that can be sampled quickly and non-invasively from breath. They can originate either from within the body (endogenous VOCs) or from external sources such as diet, prescription drugs and environmental exposure (exogenous VOCs). Endogenous VOCs are products of metabolic activity in the body and changes to these VOCs can be characteristic of specific disease processes. These may include metabolites, protein, mRNA and genes. Endogenous volatile organic compounds are produced throughout the body and are picked up and distributed in the bloodstream. From the blood they exchange into air in the lungs and are then exhaled along with respiratory droplets and atmospheric gases. Exhaled VOCs contribute to biomarkers.

In nature, the oligosaccharide chains in glycoconjugates show high specificities in biological events, the affinities of oligosaccharide chains in glycolipids and glycoproteins were enhanced by the self-assembly of glycoconjugates, which display multivalent-type clusters. Methods of formation of synthetic glycoclusters to form functionalized monomers by covalent bonds, glycodendrimers and glycopolymers, have been developed. The functionalization of monomers has been used in generating bioprobes, for example, fluorogenic monomers are useful in order to evaluate an interaction between carbohydrate molecules and a glyscosidase; biotin-avidin interaction is a very useful biological tool for biochemical and biomedical assays. There are commercially available chemical kits that provide convenient preparation of a glycomonomer and functional monomers using inexpensive reagent, such as, 4-chloromethylstyrene (CM-Str), as a starting material.

Functional monomers can also act as blocks around a template molecule that help for the orientation of a bioprobe molecule in detecting the template molecule. Using functional monomers in creating electrical sensors will therefore improve the selectivity of the sensors. In creating an electrical sensor, a functional monomer may be conjugated to a target analyte. Novel electrochemical gas sensors are generated with specific biomarkers for different diseases using functionalized graphene/Prussian-blue and molecularly imprinted polymer (MIP) on a substrate.

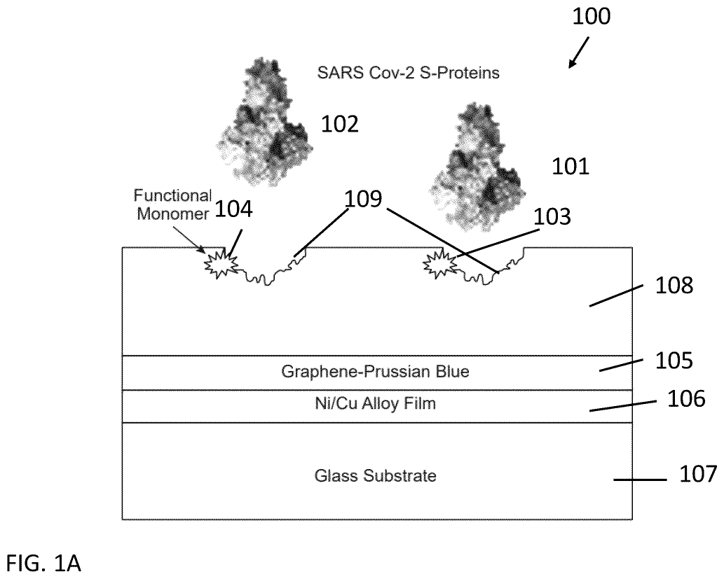

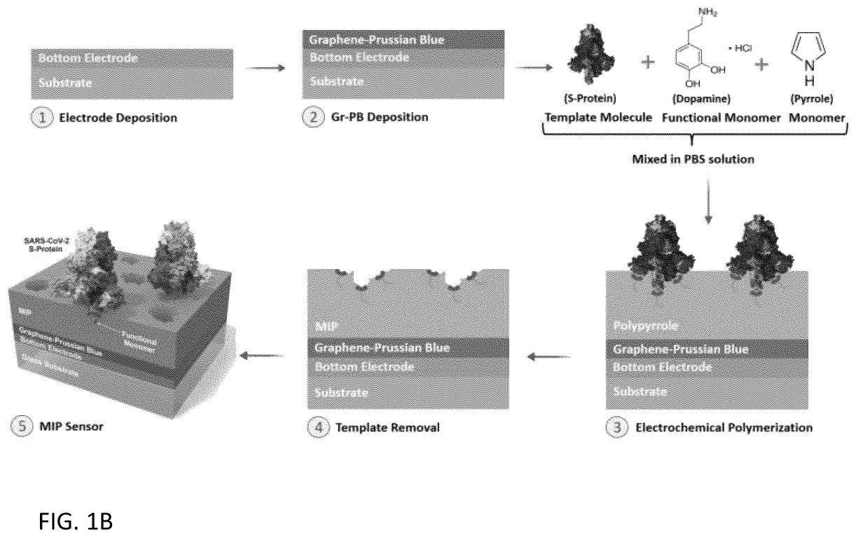

As shown in FIG. 1A, electro-sensor 100 is formed with four layers of material (105, 106, 107, 108). On the electro-conducting surface of graphene-Prussian blue 105 is a thin layer polymer 108 electrochemically deposited around template molecules or particles 101, 102, such as biomarker VOCs or SARS Cov-2-S-Proteins, that may have a strong interaction to a functional monomer 103 or 104 in the matrix polymer 108. The template molecules 101 and 102 in polymer layer 108 are then washed away from the polymerization, leaving imprint cavities 109 of the exact size and shape to the template molecules 101 and 102 in polymer matrix 108. The finally formed sensor 100 will then only detect the existence of original template molecules or particles 101 and 102 when they match into cavities 109 in shape and size and with the strong hydrogen bonding, and with interactions to monomers 103 and 104, leading to extremely high specificity. The imprinted polymers' selective recognition abilities are due to the formation of a complex between the target analyte and the selected functional monomers during the pre-polymerization step. The choice of the monomers represents a crucial point in the preparation of effective MIPs and it is based on their ability to establish interactions with the functional groups of the template molecule in a covalent or non-covalent way. Below graphene-Prussian blue layer 105 is the layer Ni/Cu alloy film 106 which is deposited on glass substrate 107 to conducting electrical change caused by the interactions between target analyte and the functional monomers.

### Method for Generating SARS-COV-2 Sensitive Electro Sensors

In one embodiment, based on prior success in using dopamine for specific detection of bovine hemoglobin, and the ability of dopamine to self-polymerize into thin adherent polydopamine (PDA) films, which could be coated on various organic and inorganic substrates, dopamine was selected as the monomer group.

Dopamine was proven to be a very good choice. Dopamine based sensors have generally characterized with the highest sensitivity and specificity ratings, as an example embodiment, dopamine-based electro sensors are generally used for measurement and testing; and methods of improvement are also based on dopamine-based electro-sensors.

Phosphate buffered saline (PBS), and full-length spike proteins with a 614G mutation (G protein), 614D mutations of SARS-COV-2 and the receptor binding domain (RBD) protein solutions are prepared. Dopamine hydrochloride was purchased from Sigma Aldrich. 100 μl RBD protein solution was made with a PBS solution containing 30 μg of the RBD protein mixed with 1 mg of dopamine hydrochloride and sonicated for 10 minutes. 100 μl RBD protein solution was mixed with an electrolyte solution of 100 ml 1M phosphate buffer solution with 690 μL pyrrole to form an electrodeposition solution. After the deposition of the graphene and Prussian-blue on the surface of a Ni/Cu glass or etched silicon substrate, the electrodeposition solution was deposited onto the graphene and Prussion-blue layer, the polydopamine polymer matrix was formed on the surface through cyclic voltammetry induced electropolymerization.

After the electropolymerization process, the electro sensor was then washed with various conditions including a 70% ethanol wash for 3 minutes and a methanol-hydrochloric acid wash for 5 seconds. The substrate effect and washing conditions may affect the sensitivity and specificity of the DA functionalized sensors. Best result was obtained by dipping the sensor into 70% ethanol solution for 3 minutes, then taking it out to dry in the air. Experiments were conducted by pipping the sensors in 70% ethanol for 30 s to 5 mins, it was found that 3 mins washing was the optimal condition to remove the template molecules and avoid destroying the MIP layer, resulting the best sensor sensitivity and specificity. Caution should be taken as the washing step can damage the MIP layer, the washing condition for different sensors needs to be carefully tested and optimized.

For the graphene-Prussian Blue (Gr-PB) layer, the ultimate fine and homogeneous Gr-PB powder was used to increase the effective surface area. Gr-PB fine powder was obtained by freeze-drying. The Gr-PB solution was centrifuged to filter out most of the water to obtain the Gr-PB thin slurry. The slurry was dispensed into a glass dish (or other similar kind of containers). The container was placed inside a vacuum chamber for freeze drying by carefully mixing liquid nitrogen with the slurry. The vacuum chamber was covered and turned on until the meter was either at max or stopped. The vacuum continued to run for about 6 hours or until the slurry was dried into fine powders.

Once the slurry was dried, the container was removed from the vacuum chamber. The resulting powders were very light-weight, fine, and homogeneous. The fined Gr-PB power was resuspend in DI water in a ratio around 4 (mg Gr-Pb): 1 (ml DI water), the solution was ready to be deposited to the top of the sensor chip substrate.

Different substrates, including Cr/Au glass, Ni/Cu Si, Ni/Cu glass, and highly doped silicon etched with HF were experimented to compare their performances. It was found that the DA gas sensors with Cr/Au glass substrates had too large a thermal coefficient of resistance for the baseline resistance measurement to be stable in the presence of random environmental temperature changes including the warmth from the tester's hand when the sample was being exposed to the sensor. The resistance of the DA gas sensors with pure Si substrate generally had a high baseline resistance ranging from approximately 1 kΩ to 1 MΩ, which shows a significant background resistance change during the measurement. The sensors produced on the Ni/Cu Si substrate had issues with responding to ice, likely due to the temperature-resistance relationship for semiconductors like silicon. Ni/Cu (Constantan) has a thermal coefficient of resistance of 10⁻⁵/K, compared to 3×10⁻³/K for Cr or Au, minimizing temperature impacts on sensor resistance and making the Ni/Cu glass (eliminating the semiconductor temperature effect of silicon as well) sensors most viable.

FIG. 1B summarizes the process. The first step of the fabrication process is the deposition of a metal electrode on a substrate such as Si or glass, and the second step is the deposition of graphene-Prussian blue on the metal electrodes. After deposition of the Graphene-Prussian blue on the surface of a metal electrode, a polypyrrole layer is formed on the Graphene Prussian blue through cyclic voltammetry-induced electropolymerization in a pyrrole-PBS solution. Functional monomers are carefully selected and added during the electropolymerization process to improve the binding between the atom groups between the template molecules and the specificity of the sensors. The selective recognition abilities of the imprinted polymers are mainly due to the formation of a complex between the analyte target and the functional monomers during the pre-polymerization step. The choice of a suitable functional monomer is based on its ability to establish a good interaction with the functional groups of the template molecule in a covalent or non-covalent way. The choice of functional monomer is critical to achieving a high-accuracy electrochemical pathogen sensor. We chose dopamine as the functional monomer for our sensors based on its prior success in the specific detection of bovine hemoglobin as well as dopamine's ability to self-polymerize into thin adherent polydopamine (PDA) films, which can then be coated on various organic and inorganic substrates. As a functional monomer, dopamine has led to pathogen sensors with the highest accuracy >99% for different variants of SARS-COV-2, compared to other functional monomers we have used, such as methacrylic acid (MAA). We have, therefore, focused our efforts on dopamine-based sensors in this work. After the electropolymerization process, the sensor was washed in ethanol to remove the template molecules and left to dry in the air before electrodes were added for sensor testing. All process steps were carefully optimized.

During the sensitivity and specificity testing, we tested the gas sensor's specificity using small vials containing room temperature deionized water (DI water), frozen DI water (ICE), phosphate buffered saline (PBS), and full-length spike proteins with a 614G mutation (G protein), respectively. The results are shown in Table I.

TABLE I
Summary of the sensitivity of sensors produced with
different substrates with water and ice as controls.

Film and Substrate Choice

| Test samples | Cr/Au Glass | Ni/Cu Si | Ni/Cu Glass |
|---|---|---|---|
| ICE | Negative | Inconsistent | Negative |
| DI water | Positive | Negative | Negative |
| G protein | Positive | Positive | Positive |

The Ni/Cu alloy used above is called Constantan. Constantan is a proprietary name for a copper-nickel alloy also known as Eureka, Advance, and Ferry. It usually consists of 55% copper and 45% nickel. Its main feature is low thermal variation of its resistivity, which is constant over a wide range of temperatures. Other known alloys with similarly low temperature coefficients are manganin (Cu [86%]/Mn [12%]/Ni [2%]). Conductors with low temperature coefficients of resistivity, such as Constantan, Manganin, etc. can reduce unwanted responses from the environment or sample temperatures, such as exhaled breath, or cold solutions.

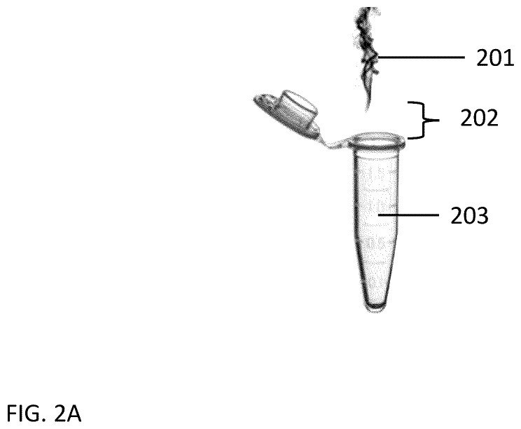

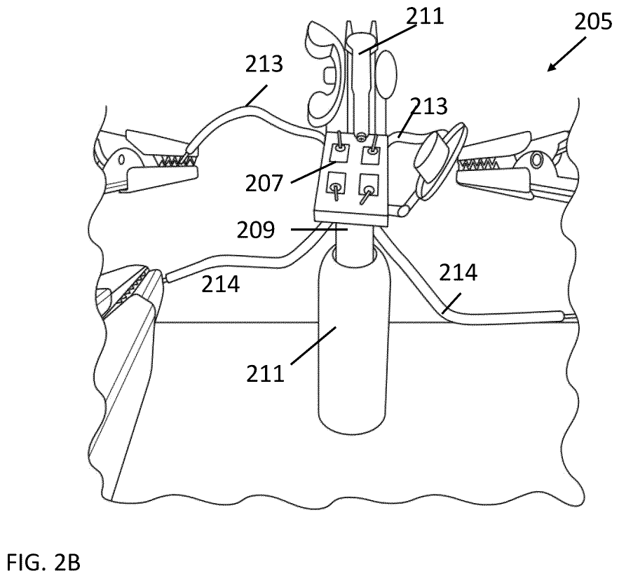

We developed an effective method to test the viability of each sensor. FIGS. 2A and 2B show our sensor testing approach in which the sensor is facing down with an open Eppendorf vial containing pathogens to be tested placed at 2˜3 mm beneath the sensor, while sensor resistance is monitored by an ohmmeter. Four-point measurement system yields more accurate results by eliminating contact resistance interference.

Example methods for fast testing SARS-COV-2 virus using bioprobe sensitized electro sensors.

FIG. 2A shows a test tube setup The bioprobe sensitized sensor 201 is facing down at the opening of an Eppendorf vial 203 that contains pathogen testing sample solution to be tested, the test tube 203 is generally placed at 2˜3 mm (202) beneath sensor 201 while sensor resistance is monitored by an ohmmeter. FIG. 2B shows an example testing setup 205. Test tube vial holder 211 motionlessly holds an Eppendorf vial 209 or other test tube vial that contains testing samples, bioprobe sensitized sensor 207 is placed facedown 2˜3 mm above testing vial 209. Electric wires 213, 214 are connected with ohmmeter to monitor sensor resistance change.

### Method for Testing SARS-COV-2 Samples

The experiments of testing SARS-COV-2 virus using a bioprobe sensitized senor were conducted by using test samples in the form of 100 μL virus solutions in 1 mL Eppendorf vials. The virus testing samples were maintained at 0 degrees Celsius before testing and were shaken in hand for 5˜10 seconds in the palm to produce aerosol inside the vial immediately before opening the vial cap and exposing the vial to the sensor. The sensor was held face-down by a clamp at a distance of 2˜3 mm above the vial opening for sensor exposure. The duration of time of exposure for each of the sensors were controlled at 10 seconds, this was the time used for the current SARS-COV-2 sensor test protocol.

The SARS-COV-2 sensitized sensors were prepared with the alpha-variant SARS-COV-2, and they were used for detecting the wild-type SARS-COV-2 virion particles and delta-variant spike proteins. For the 10 tested sensors, the analytical test results showed a sensitivity and specificity of >95% as shown in Table II, with 15 positives and negative sample test results on Covid-19 viruses for 10 sensors (#524, #526, #528, #529, #532, #534, #600, #640, #641 and #642).

Hundreds of such sensors have been tested. The sensors were sensitized with the alpha-variant RBD (with N501Y mutation) S-proteins, not the wild type, or delta variant. Then wild type SARS-COV-2 spike proteins and virion particles and Delta variant S-proteins were then used for testing. The Delta variant has mutations with the substitutions T478K and L452R in the RBD spike protein. The prepared sensors was able to still detect it. In Table II shows the test results of Gamma-ray inactivated pathogens, including SARS-COV-2, influenza A/B, with different virus loads (respectively 103, 104, 105, 106, 107 copies/mL). At the same time, the spike proteins of SARS-COV-2,hemagglutinin (HA) glycoproteins of influenza A and B viruses, and fusion (F) glycoprotein of the RSV were used as positive controls with a protein concentration ranging 0.001, 0.01, 0.1, 1, and 10 pg/μL. Negative controls included Vero E6 cell lysate, BSA, PBS, MERS, and SARS (or SARS-1) with typical concentrations of 0.1˜1 pg/μL. The sensors were tested at different temperatures from 0° C. ˜ 50° C. in simulation of the real-world breath, and various background solution controls were used, including pure water, PBS, dust, glucose, BSA to test the sensor accuracy. Our sensors are insensitive to temperatures, or other controls.

TABLE II
Summary of the sensitivity and selectivity of our sensors (“−”
testing negative; “+” testing positive)

| | Sensors IDs | | | | | | | | | |
|---|---|---|---|---|---|---|---|---|---|---|
| Test Kits | #524 | #526 | #528 | #529 | #532 | #534 | #600 | #640 | #641 | #642 |
| #1 BSA | − | − | − | − | − | − | − | − | − | − |
| #2 Wild | + | + | + | + | + | + | + | + | + | + |
| type SARS- | | | | | | | | | | |
| CoV-2 | | | | | | | | | | |
| (106 | | | | | | | | | | |
| copies/μL) | | | | | | | | | | |
| #3 Flu | − | − | − | − | − | − | − | − | − | − |
| Protein | | | | | | | | | | |
| (1 pg/μL) | | | | | | | | | | |
| #4 Flu Virus | − | − | − | − | − | − | − | − | − | − |
| (10 pg/μL) | | | | | | | | | | |
| #5 MERS | − | + | − | − | + | − | − | − | − | − |
| (1 pg/μL) | | | | | | | | | | |
| #6 PBS | − | − | − | − | − | − | − | − | − | − |
| #7 Spike | + | + | + | + | + | + | + | + | + | + |
| Protein | | | | | | | | | | |
| (1 pg/μL) | | | | | | | | | | |
| #8 Vero E6 | + | − | − | − | − | − | − | − | − | − |
| (1 pg/μL) | | | | | | | | | | |
| #9 Flu Virus | − | − | − | − | − | − | − | − | − | − |
| (1 pg/μL) | | | | | | | | | | |
| #10 Wild | + | + | + | + | + | + | + | + | + | + |
| type SARS- | | | | | | | | | | |
| CoV-2 (10⁴ | | | | | | | | | | |
| copies/μL) | | | | | | | | | | |
| #11 Delta | + | + | + | + | + | + | + | + | + | + |
| variant S- | | | | | | | | | | |
| proteins (10 | | | | | | | | | | |
| pg/μL) | | | | | | | | | | |
| #12 Delta | + | + | + | + | + | + | + | + | + | + |
| variant S- | | | | | | | | | | |
| proteins | | | | | | | | | | |
| (1 pg/μL) | | | | | | | | | | |
| #13 Wild | + | + | + | + | + | + | + | + | + | + |
| type SARS- | | | | | | | | | | |
| CoV-2 | | | | | | | | | | |
| (10⁵copies/mL) | | | | | | | | | | |
| #14 Wild | + | + | + | − | + | + | + | + | + | + |
| type SARS- | | | | | | | | | | |
| CoV-2 (10³ | | | | | | | | | | |
| copies/mL) | | | | | | | | | | |
| #15 Flu | − | − | − | − | − | − | − | − | − | − |
| Virus | | | | | | | | | | |
| (0.1 pg/μL) | | | | | | | | | | |

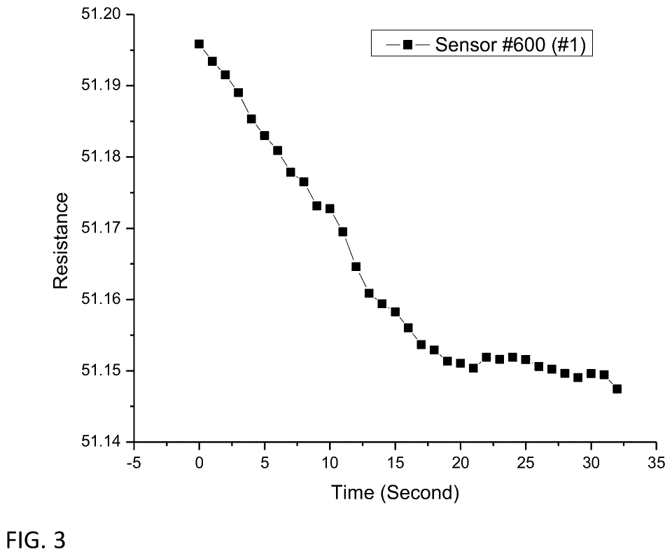

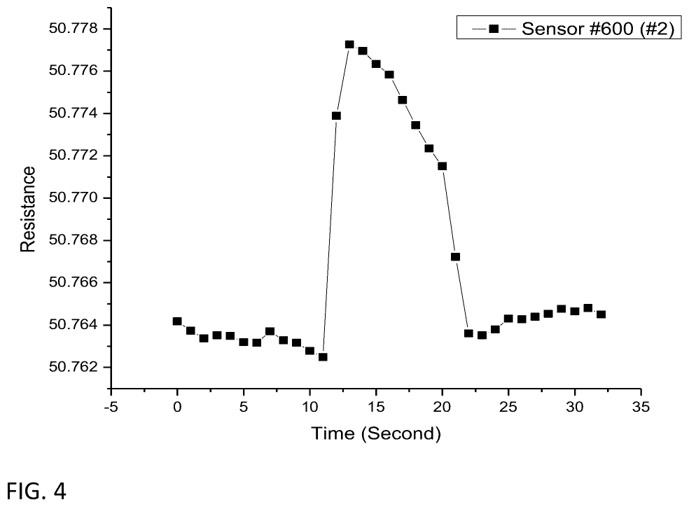

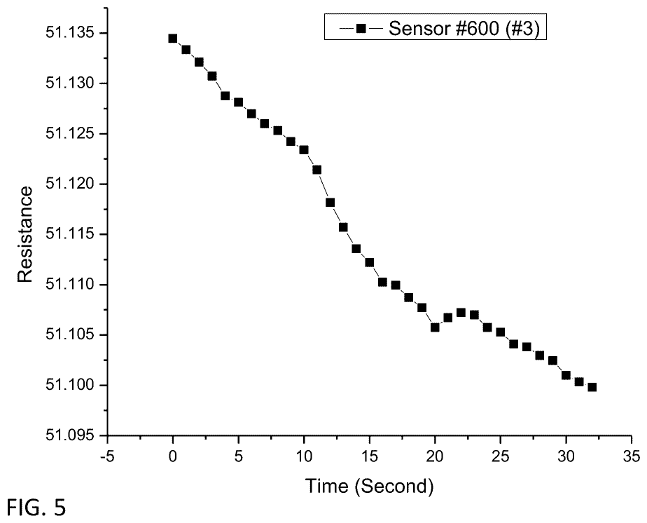

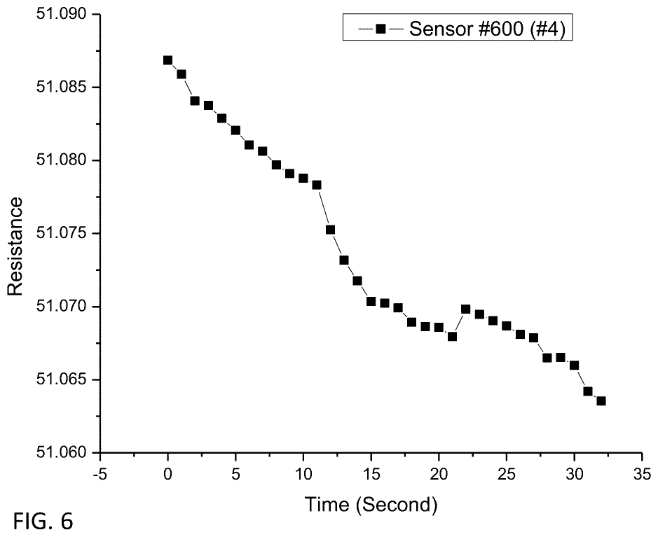

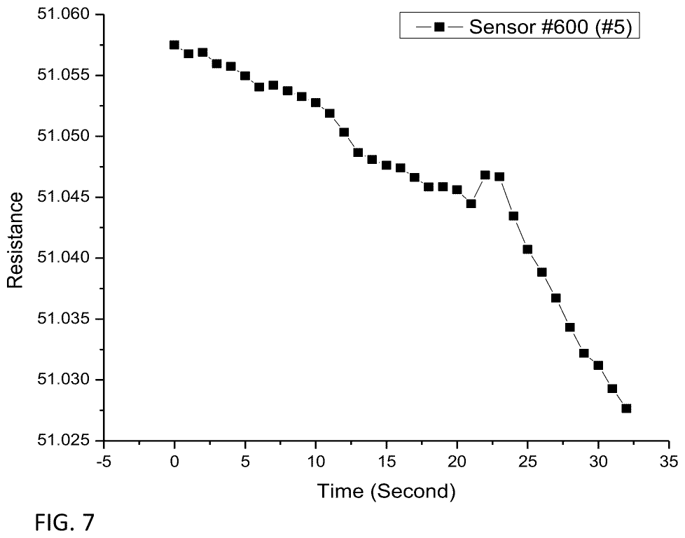

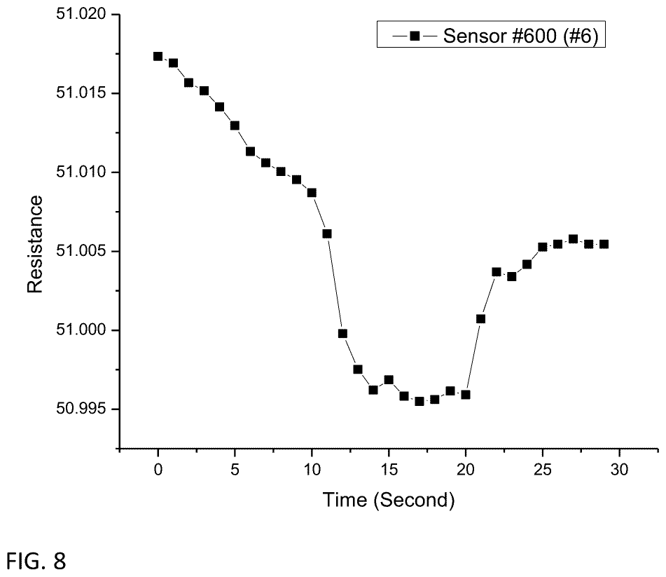

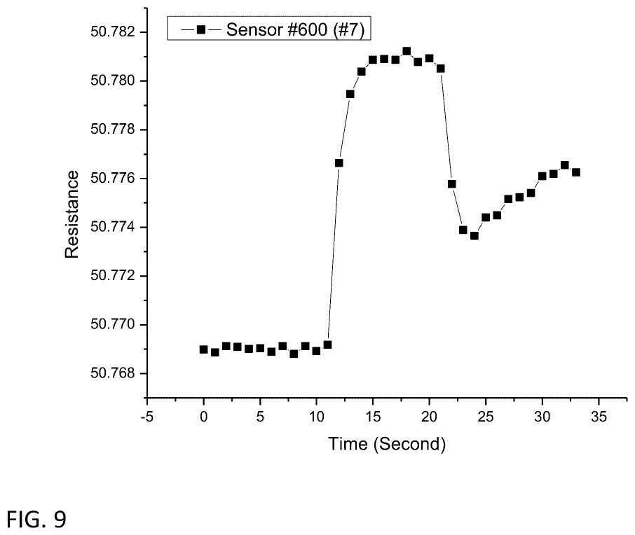

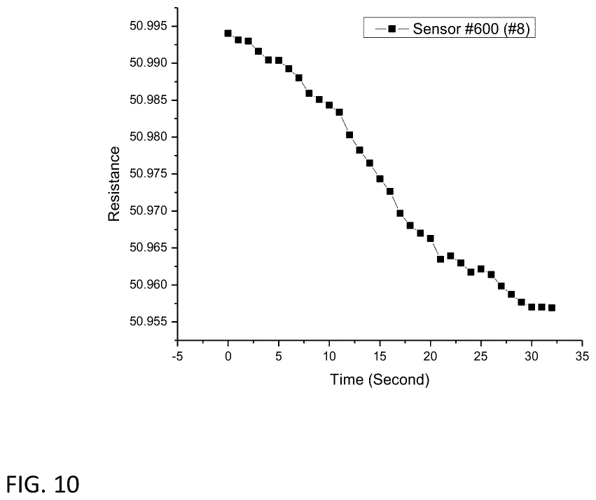

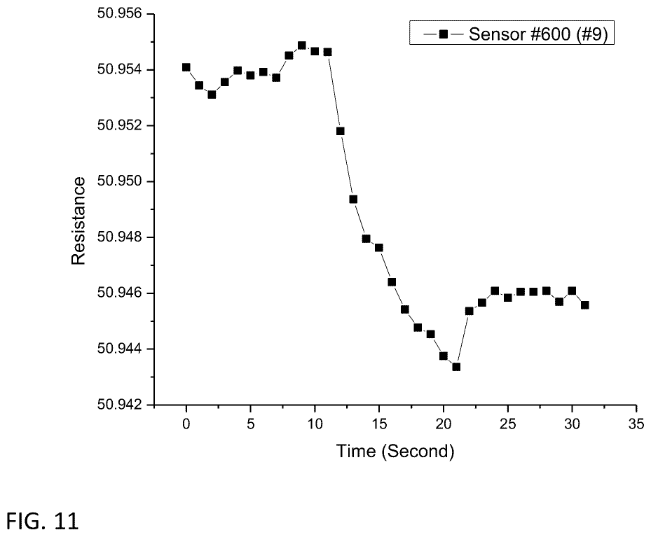

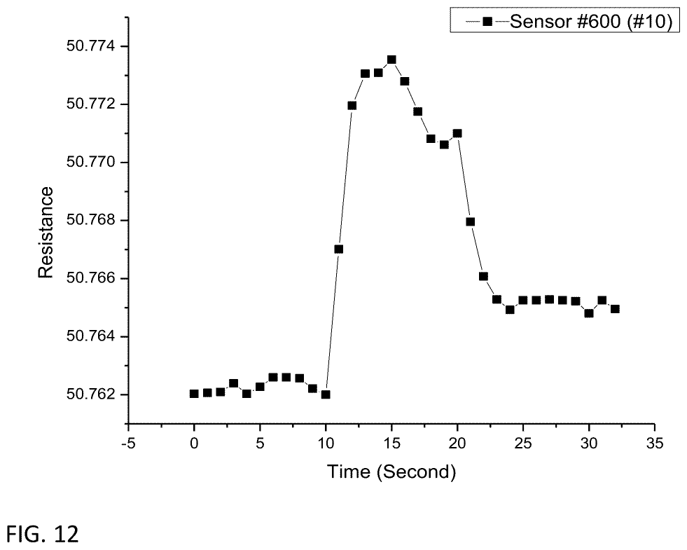

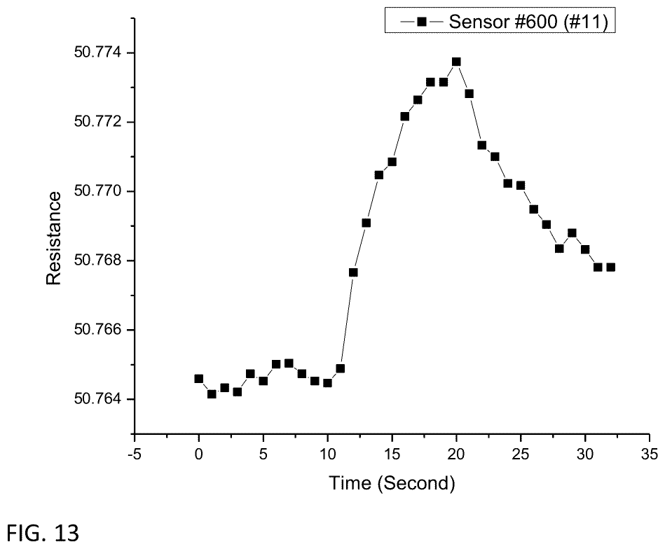

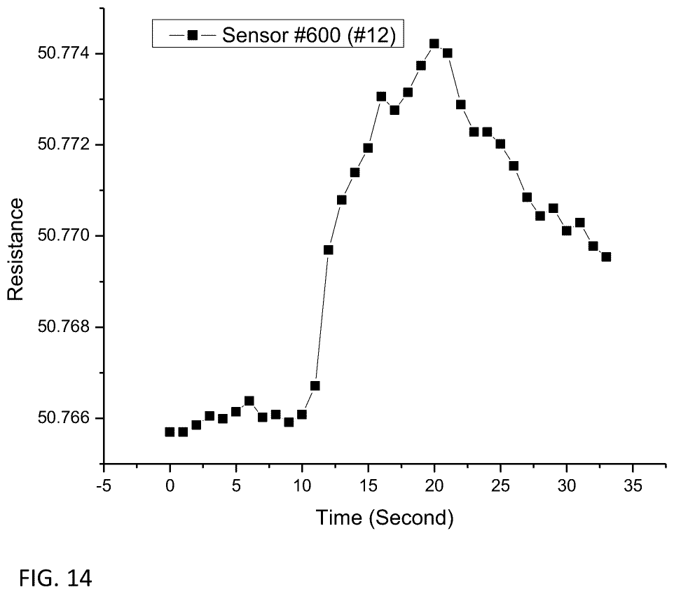

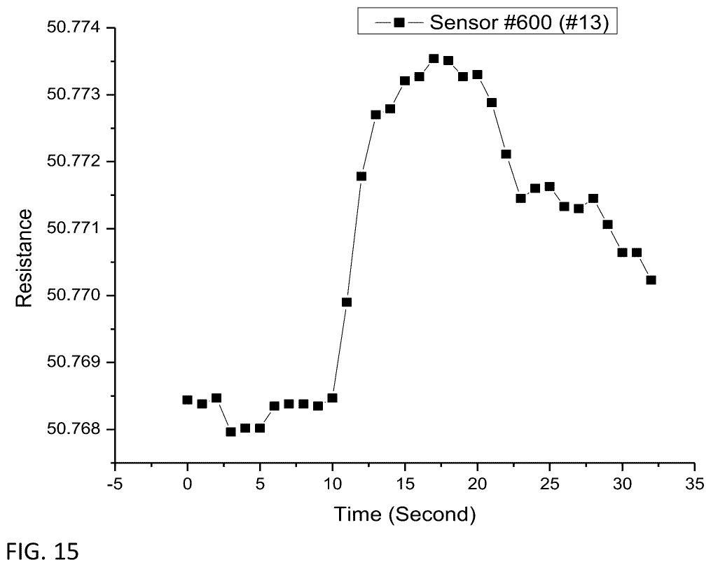

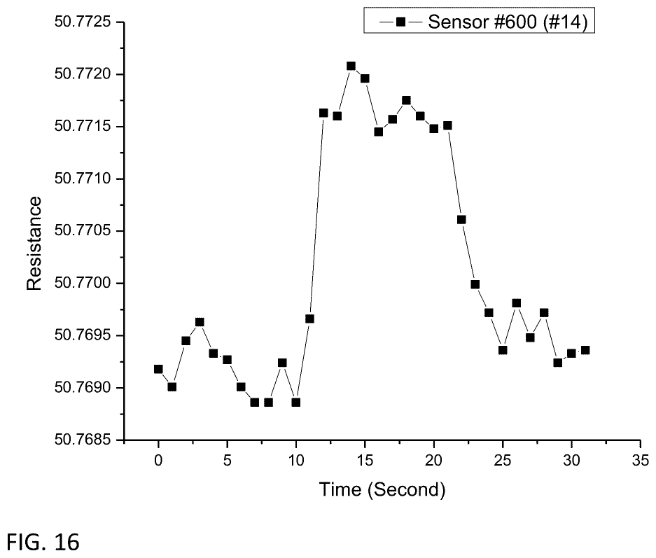

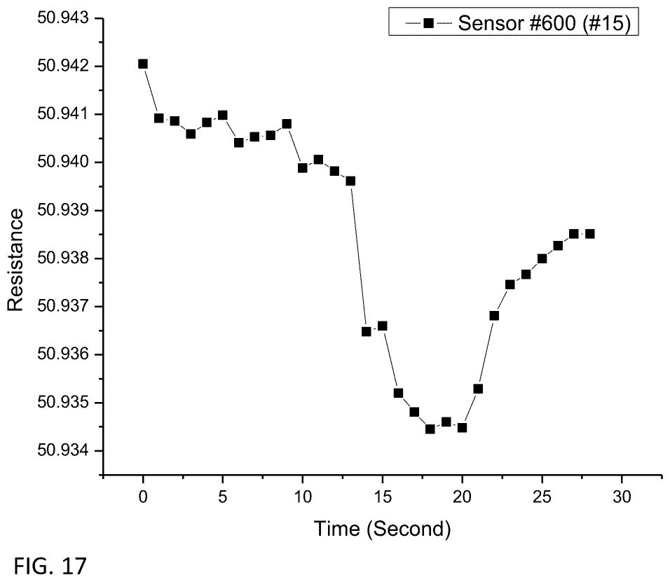

FIGS. 3-17 show the measurement of resistance changes with respect to time in response to various test samples. FIG. 3 shows the negative response to bovine serum albumin protein (BSA) control. FIG. 4 shows the positive sensor response to wild-type SARS-COV-2 (106 copies/μL). FIG. 5 shows the negative response to Flu Protein (1 pg/μL) (106 copies/μL). FIG. 6 shows the negative response to Flu Virus (10 pg/μL). FIG. 7 shows the negative response to MERS (1 pg/uL). FIG. 8 shows the negative response to PBS. FIG. 9 shows the positive response to Spike Protein (1 pg/uL). FIG. 10 shows the negative response to Vero E6 (1 pg/uL). FIG. 11 shows the negative response to Flu Virus (1 pg/μL). FIG. 12 shows the positive response to wild type SARS-CoV-2 (104 copies/uL). FIG. 13 shows the positive response to Delta variant Spike Protein (10 pg/uL). FIG. 14 shows the positive response to Delta variant Spike Protein (1 pg/uL). FIG. 15 shows the positive response to wild type SARS-COV-2 (105 copies/mL). FIG. 16 shows the positive response to wild type SARS-COV-2 (103 copies/mL). FIG. 17 shows the negative response to Flu Virus (0.1 pg/uL).

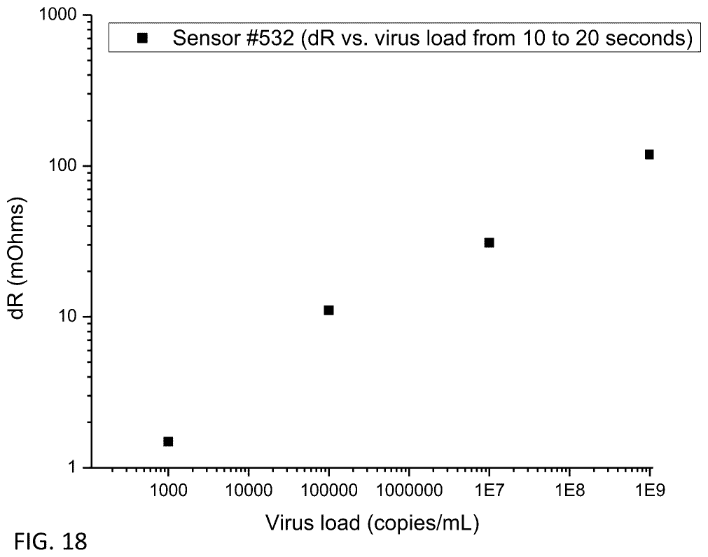

Sensor resistance changes after aerosol exposure: deltaR (dR in mOhms) vs. virus load from 10 to 20 seconds show a similar resistance change trend for all sensors, that is, a lower deltaR with the reduction of virus load in the solution. FIG. 18 shows virus copy correlation that deltaR of ˜1 mΩhms is the limit of detection of resistance, and a limit of detection better than 1000 copies/mL for SARS-Cov-2.

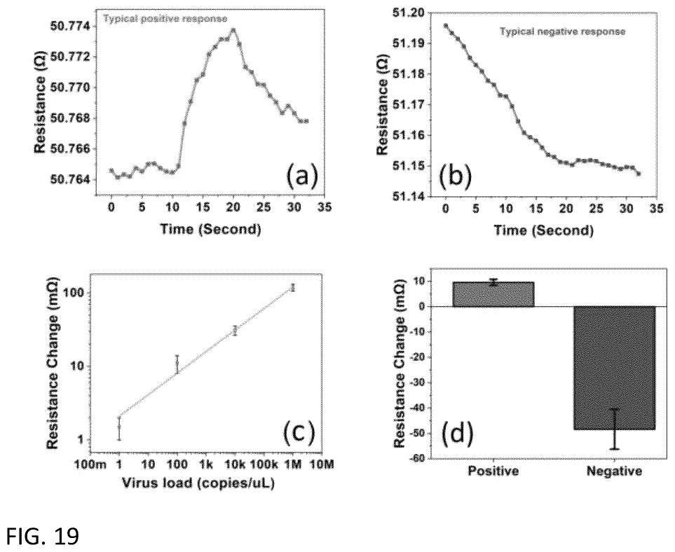

FIG. 19 shows a typical positive test result obtained using the 10:10:10 method for the Delta variant Spike Protein (10 pg/mL). As is seen in FIG. 19, panel (a), due to the random noise (which is nearly 1 mΩ RMS) the resistance is not exactly equal to zero during the initialization phase. During the exposure phase, the resistance sharply increases with time until it reaches its maximum at t=20 s, at which time the exposure is cut off. The resistance then drops with time during the recovery phase. The response becomes totally different when the sample does not contain the pathogen. FIG. 19, panel (b) shows the negative test result for bovine serum albumin protein (BSA), which contains no pathogen. As is seen in panel (b), for a negative test the resistance profile is a decaying function of time, on a time-averaged basis. In fact, negative response is mainly dominated by the noise during all three phases. The limit of detection of these pathogen sensors can be estimated when the resistance change is equal to the noise level at 1 mΩ leading to a signal-to-noise ratio of 1 or, equivalently, 0 dB. The limit of detection for the sensor shown in FIG. 19 is between 0.1 to 1 copies/μL (i.e., 0.1-1 fg/ml), which is comparable to the conventional RT-PCR. It is important to note that, for the positive test cases, the load plays a key role on the ohmic resistance. It is speculated that the resistance-change (ΔR) increases with the pathogen loads of the gamma-ray inactivated wild-type SARS-COV-2 virus. The log-log plot in panel (c) shows that this is indeed the case. In fact, based on the data presented in this figure, ΔR increases with the load in a nonlinear fashion. The best fit turned out to be of the power-law form: ΔR=k×(virus_load)ⁿ where the coefficient (k) and the exponent (n) are found to be equal to 0.2748±0.1064 and 0.2935±0.0209, respectively—the uncertainties associated with (k,n) have been obtained from linear regression analysis on the log-transformed data. The error bars in Panel (c) are a clear indication of the errors associated with the electrical measurements. Panel (d) is a clear comparison in resistance changes for positive and negative samples.

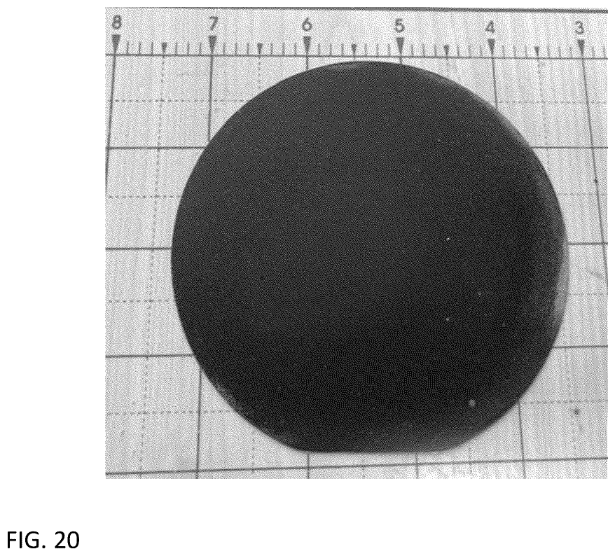

We experimented and analyzed with sensors made with active carbon with carboxymethyl cellulose (AC-CMC) layer. AC-CMC has been shown in the literature to have an incredibly high effective surface 2600 m²/g, making it an excellent candidate for increasing the surface area of the sensor. By increasing the effective surface area, more template molecules can be implanted and removed from the sensor surface, thus increasing the positive signal produced. The fabrication of the AC-CMC layer was based primarily on the paper by Garakani, M. A. et al, “Scalable spray-coated graphene-based electrodes for high-power electrochemical double-layer capacitors operating over a wide range of temperature.” Energy Storage Materials 34, 1-11. Link for the paper: doi:https://doi.org/10.1016/j.ensm.2020.08.036 (2021), the entirety of which is therefore incorporated by reference. FIG. 20 displays a full-size wafer after the deposition of AC-CMC using spray coating process that can subsequently be broken up into numerous uniform sensors. We were able to manufacture the wafer-scale for these SARS-COV-2 sensors where active carbon successfully replaced graphene and achieved comparable sensor performance but improved manufacturability.

During the manufacturing process, 4″˜12″ diameter wafers were spray coated of graphene-Prussian blue with subsequent wafer-level electrodeposition to form the MIP layer using the whole wafer to realize the mass manufacturing of these sensors. One 6″ wafer produced ˜700 sensors, as one sensor is 5 mm×5 mm. Wafer-scale spray deposition of graphene-Prussian blue layer and wafer-scale electrochemical deposition of the MIP layer thus can be used for mass manufacturing of these sensors.

Packaging of conventional integrated circuit chips involves wafer dicing and cleaning. A new approach to manufacturability and subsequent packaging of these pathogen sensors does not go through conventional wafer dicing and cleaning steps, a semiconductor integrated circuit chips is only a half-diced wafer from behind, leaving 100˜150 μm thick wafers and the metal electrode film undiced, so that the wafer maintains its integrity for the wafer-scale sensor manufacturing process. A chip dicing process is no longer needed after the sensors are made. We have tried to dice the wafers and leave different thickness. We identified that for 1 to 12 inch si or glass wafers, a thickness of 100˜150 μm thickness left undiced in the dicing trench allows excellent integrity of the wafers needed for wafer-scale manufacturing, and at the same time, the individual sensors can be detached from the wafer with a tool such as a vacuum pick or tweezer, without the need to dice the sensors chips from the wafers.

The wafer was half diced from back side of the wafer to form the trench for breaking the sensor into small chips with the sizes of small squares of 100 μm˜100 mm (e.g. 5 mm×5 mm), leaving 50˜500 μm (e.g. 100˜150 μm) thickness of the wafer un-diced for maintaining the full-wafer integrity, but easy to break the wafter for individual sensors from the back side into a chip after the wafer sensor is fabricated.

### Artificial Intelligence Methods for COVID-19 Diagnosis

In this section, we design and evaluate several detection schemes based on machine learning. To this end, we rely on an experimental dataset of L time series that were acquired as noted in the prior sections. We denote by {xᵢ}=ᵢ₌₁^{L} the ith experimental time series of the dataset. As seen below, each test set is of length 30, corresponding to a 30-second observation sampled at 1 Hz. Each experimental time series is associated with a known binary label yᵢ ∈{0, 1} that takes value 0 or 1 in the absence or presence of SARS-CoV-2 pathogens in the aerosol being tested, respectively. We design a classifier, represented by a function ƒ:R→1 that associates every input time series x of length n with a binary prediction corresponding to inference on the presence of SARS-COV-2 pathogens in the tested aerosol. An efficient classifier should achieve low empirical risk, such that the number of classified entries in a test dataset of L time series given by Σᵢ₌₁^{R}(ŷᵢ, yᵢ)=Σᵢ₌₁^{R}(ƒ(xᵢ), yᵢ) is small, where the function R equals 0 if ŷᵢ=yᵢ and 1 otherwise. In practice, the risk function R can be weighted to balance between the two types of the probability of error: P[ƒ(x)=0|pathogens] and P[ƒ(x)=1|no pathogen]. In the sequel, three categories of classifiers are considered. We first consider two model-driven classifiers based on the wavelet decomposition. These classical methods rely on prior model assumptions on the structure of the acquired time series. Moreover, they come with the advantage of needing a small number of samples to train the classifier. The wavelet decomposition assumes that there is an underlying deterministic function mapping time to the resistance that linear combinations of wavelet functions can describe. The Gaussian mixture model assumes that the resistance can be described by a random variable with a distribution that is a mixture of Gaussian probability density functions.

This approach has been demonstrated to lead to 97.8% accuracy for SARS-COV-2 based pathogen sensors. The curve-fitting approach relies on artificial intelligence's (AI's) capacity to identify COVID-19 pathogen from sensor data. The diagnosis is produced by the algorithm that we have developed in Java language using the raw data from the biomarker breathalyzer sensors. We determined the regression lines for each of the 10-second period using standard deviation and Pearson correlation coefficient. The initialization, exposure, and recovery phases are the three-time windows that make up the sensor response. The algorithm aims to determine if the sensor resistance during the exposure phase was significantly affected to warrant a positive diagnosis as biomarkers increase the sensor's resistance. The algorithm utilizes a total of four conditions to assess whether the aerosol (to the sensor exposed) influenced the sensor resistance that is significant enough or not. The first condition determines if the exposure phase's regression slope is higher than the initialization phase's regression slope. The second condition determines if the exposure phase's regression slope is greater than the recovery phase's regression slope. The third condition determines if the exposure phase's regression slope is greater than zero. The final condition determines whether the exposure phase's range is larger than the initialization phase's range. Conditions one and two check whether the rise in resistance during the exposure phase was greater than that in the initialization and recovery phases, indicating an impact created by the substrate on the sensor. The third condition ensures that the magnitude of change is directed in the positive direction during the exposure phase. Finally, the fourth condition determines whether the changes experienced during the exposure phase are significant compared to the changes seen during the initialization phase. If the changes are not more significant, they can be marked down as noise and discarded. Clearly, the analysis described in this sub-section demonstrates that the pathogen sensors are statistically informative for detecting SARS-COV-2 pathogens in aerosols.

The models are tested on a dataset of 135 experimental time series, among which 63 are positive and 72 are negative. A cross-validation method is adopted to train and test the models. This consists of firstly partitioning the time series into five distinct subsets of 27 time series at random. Next, one subset is discarded and left for validation for each of the five training batches, while the four others are retained for training purposes. The accuracy and specificity of the classifier are defined as the mean statistics over the batches. The wavelet-based and Gaussian mixture model classifiers are implemented under MATLAB. The LSTM neural network is implemented on one computer with Nvidia Geforce RTX 3060 Graphical Processing Units and 16 GB of memory. PYTORCH 1.10.0 is employed as the deep learning framework. Consistent with the experiments for the other methods, we use the same dataset for training and testing. Note that, to make a fair comparison with the other methods, there are no samples for validation, i.e., the samples are either for training or testing. For the training process of the deep learning-based method, the learning rate and the momentum are set to 0.01 and 0.9, respectively. The batch size is selected as 32, considering the size of the dataset, and the dropout rate is set to 0.2 for the first three FCLs. The designed network is trained for 2000 epochs. The performance of the two mathematical models (wavelet-based vs. deep-learning-based) in comparison with the decision made by human brain via visual inspection. For comparison, in Table III, the sensor system of this application developed by Winchester Technologies is compared with other commercially available systems. As is seen in this list, the deep learning-based classification shows a better trade-off between true positive and true negative rates but at a price of more complexity than the supervised learning methods. Still, it should be conceded that in circumstances where the computational training power is limited, the wavelet-based classifier remains a good compromise.

TABLE III
A comparison between the novel sensor developed in this application
with those already available on the market for Covid-19 test.

| | Approach | Sensitivity | Specificity | Time | | Body Fluids | Vendors |
|---|---|---|---|---|---|---|---|
| RT-PCR | RNA | 95-100% | 95-100% | 3 | hr | Respiratory specimens | LabCrop, |
| | | | | | | | Roche, etc. |
| Antibody | IgM, IgG | 80-100% | 90-100% | 15-30 | min | Blood | Cellex, etc. |
| Antigen | RNA | 20-71% | 85-100% | 15-30 | min | Blood | UCSD |
| CRISP- | RNA | 95% | 100% | 40 | min | Respiratory specimens | Sherlock |
| based | | | | | | | Biosciences |
| LAMP- | RNA | 97% | 100% | 30 | min | Respiratory specimens | MicrosensDx |
| based | | | | | | | |
| Saliva- | RNA | 91% | 98% | 45 | min | Saliva | Yale School |
| based | | | | | | | of Public |
| | | | | | | | Health |
| Breath- | GC-MS | 91% | 99% | 5-10 | min | Air | Inspect-IR, |
| based | VOC | | | | | | Breathomix |
| Our sensor | S-RBD | 98.40% | 100% | 10 | sec | Aerosol/Air | Winchester |
| | Protein | | | | | | Technologies |

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

Additional general background, which helps to show variations and implementations, may be found in the following publications, all of which are hereby incorporated by reference herein for all purposes:

Center for Systems Science and Engineering at Johns Hopkins University. Coronavirus COVID-19 Global Cases. https://www.military.com/daily-news/2020/04/16/hundreds-sailors-carrier-roosevelt-have-covid-19-fewer-half-had-symptoms.html.

Powell A. 2020, “Harvard experts say Massachusetts may need 1.4 million tests.” Harvard Gazette.

Sheila Kaplan K T. 2020, “Despite Promises, Testing Delays Leave Americans “Flying Blind.” The New York Times.

Gudbjartsson D F, et al. 2020, “Spread of SARS-COV-2 in the Icelandic Population.” N Engl J Med. DOI: 10.1056/NEJMoa2006923.

Zou L, et al. 2020, “SARS-COV-2 Viral Load in Upper Respiratory Specimens of Infected Patients.” N Engl J Med 382:1177-1179.

van Doremalen N, et al. 2020, “Aerosol and Surface Stability of SARS-COV-2 as Compared with SARS-COV-1.” N Engl J Med.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle. The claims as filed are intended to be as comprehensive as possible, and NO subject matter is intentionally relinquished, dedicated, or abandoned.

What is claimed is:

1. A biomaterial sensitized sensor for testing a virus, comprising:
a layer of substrate;
a layer of conducting metal electrode being disposed on the surface of the layer of substrate;
a layer powders of selected from the group of graphene and active carbon coated with potassium ferrocyanide, being disposed on the surface of the layer of conducting metal electrode;
a layer of molecularly imprinted polymer (MIP) being disposed on the surface of the layer of powders;
wherein the layer of MIP is made by imprinting with a template virus material in mixture with a functional monomer chemical material through a step of washing.

2. The biomaterial sensitized sensor of claim 1, wherein the layer of substrate is either glass or silicon, and the layer of conducting metal electrode is made of a metal selected from the group of Ni—Cu alloy and Cu—Mn—Ni alloy.

3. The biomaterial sensitized sensor of claim 1, wherein the layer of powders is made from powders obtained by a freeze-drying process for achieving a sufficient large specific surface area and sufficient high sensitivity.

4. The biomaterial sensitized sensor of claim 1, wherein the layer of powders is disposed using spray coating technique.

5. The biomaterial sensitized sensor of claim 1, wherein the layer of MIP is imprinted by dipping the pre-washing sensor in 70% ethanol in water from 3 seconds to 5 minutes to wash away the template virus material.

6. The biomaterial sensitized sensor of claim 5, wherein the dipping time is 3 minutes.

7. The biomaterial sensitized sensor of claim 1, wherein the functional monomer chemical material is dopamine or dopamine hydrochloride.

8. The biomaterial sensitized sensor of claim 7, wherein the polymer layer is formed on the surface of the layer of graphene through cyclic voltammetry induced electropolymerization.

9. The biomaterial sensitized sensor of claim 1, wherein the virus template material comprises a protein or pathogen particles selected from the group of SARS-1, SARS-COV-2, Delta variant SARS-COV-2, Omicron variant SARS-COV-2, flu A, flu B, RSV, MERS HPAI, BSA, and miRNA.

10. The biomaterial sensitized sensor of claim 1 is manufactured by using a 1 to 12-inch diameter silicon or glass wafer as the substrate layer.

11. A method of testing a pathogen in a sample, comprising the steps of:
connecting the sensor of claim 1 to an ohm-meter or a multimeter for electric resistance measurement;
recording the electric resistance of the sensor for 10 seconds;
exposing the senor to an aerosol of the sample for 10-20 seconds;
stopping exposing of the sensor from the sample;
recording the electric resistance of the sensor for another 10 seconds after stopping the exposure to the sample;
processing the recorded electric resistance of the sensor using the Artificial Intelligence model to arrive a conclusion about the existence of the pathogen in the sample.

12. The method of claim 11 is conducted with a 2-point or 4-point electric resistivity measurement system.

13. The method of claim 11, wherein the sample has a volume about 10 μl to 1 dL, placed in a vial, and the sensor is facedown placed about 0.5-50 mm above the opening of the vial.

14. The method of claim 11, wherein the testing temperature ranges from −20° C. to 50° C.

15. The method of claim 11, wherein a curve-fitting AI algorithm is used for determining whether the recorded electric resistance changes indicate a positive or negative test for a particular pathogen.

16. The method of claim 15, wherein the AI algorithm contains curve-fitting standards for testing a group of pathogens and proteins.

17. A method for manufacturing a wafer sized sensor using a wafer of claim 1, comprising the steps of:
depositing a layer of electrode film with low thermal coefficient of resistivity on the front surface of a 1 to 12 inch wafer made of silicon or glass;
half dicing the wafer from back side of the wafer to form trench squares of 100 μm to 100 mm in sizes, leaving 50 to 500 μm thickness of the wafer un-diced for maintaining the full-wafer integrity;
spray-coating a layer of powder made of Prussian blue coated graphene or active carbon onto the surface of the electrode film;
depositing an electrodeposition solution on the surface of the powder layer;
said electrodeposition solution composed of a complex solution made with a ratio of 100 μl of a PBS solution containing a sufficient amount template protein or pathogen particle mixed with sufficient amount of dopamine or dopamine hydrochloride, sonicated in an electrolyte solution of 100 ml 1M phosphate buffer solution with 690 μL pyrrole, to form a polymer layer and a pre-washed senor-wafer;
after formation of the polymer layer, dipping the pre-washed sensor-wafer into a solvent solution for 3 seconds to 5 minutes; and
vacuum picking or tweezer a sensor chip off the wafer without dicing the wafer again.

18. The method of claim 17, wherein the powder is obtained by a freeze-drying method.

19. The method of claim 17, wherein the electrode film is made of a metal selected from the group of Ni-Cu alloy and Cu-Mn-Ni alloy.

20. The method of claim 17, wherein the template protein or pathogen particle is selected from the group of SARS-1, SARS-COV-2, Delta variant SARS-COV-2, Omicron variant SARS-COV-2, flu A, flu B, RSV, MERS HPAI, BSA, and miRNA.
